SEQUENCE LISTING (2 sequences)

SEQ ID NO: 1 - DNA, 20 bp, synthetic construct
  source 1..20: mol_type = other DNA
gcaagaggtgcggaagatta

SEQ ID NO: 2 - DNA, 21 bp, synthetic construct
  source 1..21: mol_type = other DNA
ggatggcgaagctaagatcaa

(19) United States
(12) Patent Application Publication
(10) Pub. No.: US 2023/0035004 A1
(43) Pub. Date: Feb. 2, 2023

(54) THERAPEUTIC COMPOSITIONS CONTAINING BACTERIAL OUTER MEMBRANE VESICLES AND USES THEREOF

(71) Applicant: NUtech Ventures, Lincoln, NE (US)

(72) Inventors: Kari Heck, Lincoln, NE (US); Amanda Ellen Ramer-Tait, Lincoln, NE (US); Angela Kaye Pannier, Lincoln, NE (US)

(21) Appl. No.: 17/815,729

(22) Filed: Jul. 28, 2022

Related U.S. Application Data
(60) Provisional application No. 63/226,523, filed on Jul. 28, 2021.

(51) Int. Cl. A61K 9/50; A61K 31/713; A61K 9/51

(52) U.S. Cl. CPC A61K 9/5068; A61K 31/713; A61K 9/5161

(57) ABSTRACT

This document provides therapeutic compositions, methods of making, and methods of using the described therapeutic compositions that include outer membrane vesicles.

### CROSS-REFERENCE TO RELATED APPLICATION

This application claims the benefit of priority to U.S. Patent Application No. 63/226,523, filed on Jul. 28, 2021, which is incorporated herein by reference in its entirety.

### FEDERALLY SPONSORED RESEARCH OR DEVELOPMENT

This invention was made with government support under EB025760 and GM107001 awarded by the National Institutes of Health and under 2017-67015-26456 awarded by the U.S. Department of Agriculture. The government has certain rights in the invention.

### INCORPORATION BY REFERENCE

AXML file named 24742-0131001_SL_ST26.xml. The XML file, created on Jul. 27, 2022, is 2,850 bytes in size. The material in the XML file is hereby incorporated by reference in its entirety.

### TECHNICAL FIELD

This document provides therapeutic compositions and methods of making and use thereof related to outer membrane vesicles (OMVs). For example, this document provides therapeutic compositions that can include a therapeutic moiety and are loaded in or on commensal bacterial OMVs.

### BACKGROUND

Oral delivery of therapeutic moieties, for example, nucleic acids, proteins, or small molecules, can treat and prevent a variety of diseases. Benefits of oral administration include that it is non-invasive, associated with high patient compliance and convenience, and provides local and systemic delivery options. However, oral delivery of therapeutics is complicated by chemical (e.g., acidic pH) and physical barriers (e.g., epithelial layer) within the gastrointestinal (GI) tract. Materials that have been investigated for oral delivery systems (e.g., chitosan, gelatin, poly(lactic-co-glycolic acid, etc.) often fail to protect the therapeutic moiety cargo, such as DNA, from the low pH and DNA-degrading enzymes present in the GI tract. This can result in improper or incomplete degradation of the exterior protective coating of the therapeutic moiety, resulting in undesirable release kinetics of the enclosed therapeutic moiety and variability in the outcome, for example, variability in transgene production from therapeutic DNA moiety. Additionally, there are limited cellular targeting mechanisms to promote uptake of the therapeutic by host cells, such as GI tract epithelial cells. Thus, oral delivery of therapeutic moieties, such as DNA, requires a vehicle capable of protecting the cargo from degradation during GI transit, targeting uptake by intestinal cells to facilitate production of therapeutic amounts of transgene, and regulating immune response profiles.

### SUMMARY

This document provides therapeutic compositions, methods of making, and methods of using the described therapeutic composition. The therapeutic compositions include commensal bacterial OMVs and can be loaded with a therapeutic moiety (e.g., a therapeutic proteins or DNA construct to express a therapeutic protein).

Described herein are therapeutic compositions including a commensal bacterial outer membrane vesicle (OMV), and a therapeutic moiety.

In some embodiments, the commensal bacterial OMV is from a bacterium that is typically a resident of the gut. In some embodiments, the commensal bacterial OMV is from is a resident bacterium of the gut that is not typically pathogenic. In some embodiments, the commensal bacterial OMV is from a bacterium that is not intergeneric.

In some embodiments, the commensal bacterial OMV is from a gram-negative bacterial species. In some embodiments, the commensal bacterial OMV is from a bacterial genera selected from Akkermansia, Bacteroides, Enterococcus, Escherichia, Klebsiella, Parabacteroides, Prevotella, Pseudomonas, Staphylococcus, Streptococcus, or Veillonella. In some embodiments, the commensal bacterial OMV is an Escherichia coli bacterial species. In some embodiments, the E. coli bacterial species is selected from DH5-alpha, 541-15, 568-2, T75, LF82, 79, 88, 117, 128, 132, 142, 143, 147, 149, UM-146, HM427, HM428, HM452, HM454, HM455, HM456, HM463, HM484, HM488, HM489, HM615, 4F, 13I, 30A, 150F, NRG857c.

In some embodiments, the commensal bacterial OMV has a diameter of about 50 nm to about 300 nm.

In some embodiments, the commensal bacterial OMV is a purified or isolated commensal bacterial OMV. In some embodiments, the commensal bacterial OMV is enriched compared to the commensal bacterial culture.

In some embodiments, the therapeutic moiety is selected from a nucleic acid, a protein, a chemical, a drug, or a small molecule. In some embodiments, the nucleic acid is a RNA molecule (e.g., a silencing RNA, a non-coding RNA or a messenger RNA). In some embodiments, the nucleic acid encodes a protein (e.g., a therapeutic protein, a transcriptional activator, or a transcriptional repressor). In some embodiments, the therapeutic protein is selected from a cytokine, an antigen, an antibody, a biologic, a growth factor, an enzyme, a differentiation factor, an immune modulating factor, or a vaccination protein. In some embodiments, the nucleic acid is a viral vector. In some embodiments, the drug is an antibiotic, a biologic, a steroid, a chemotherapeutic, or an immunosuppressant.

In some embodiments, the composition further includes a pharmaceutically acceptable carrier. In some embodiments, the therapeutic composition is formulated for oral administration. In some embodiments, the therapeutic composition is formulated as a tablet, suspension, a capsule, or other suitable formulation for oral delivery.

Also provided herein are methods of making a therapeutic composition including the steps of a) providing a commensal bacterial outer membrane vesicle (OMV), and b) introducing a therapeutic moiety into and/or onto the commensal bacterial OMV. In some embodiments, the providing step comprises filtering or centrifuging a culture of a commensal bacterial species to obtain the OMVs. In some embodiments, the commensal bacterial OMV is purified or isolated. In some embodiments, the commensal bacterial OMV is enriched.

In some embodiments, the introducing step comprises electroporation, surface adsorption, or extrusion. In some embodiments, any of the methods described herein further include the step of visualizing the commensal OMV using transmission electron microscopy (TEM). In some embodiments, any of the methods described herein further include the step of measuring the average diameter of the OMVs.

Also provided herein are methods of delivering a therapeutic moiety to an individual including administering any of the therapeutic compositions described herein to an individual in need thereof.

In some embodiments, the individual suffers from inflammatory bowel disease, ulcerative colitis, Crohn's disease, colon cancer, irritable bowel syndrome, lactose intolerance, celiac disease. In some embodiments, the individual receives a nucleic acid-based vaccination. In some embodiments, the administering comprises oral delivery, intranasal administration, or rectal administration. In some embodiments, the administering is repeated a plurality of times.

In some embodiments, any of the methods described herein further include monitoring for the presence, absence and/or amount of the therapeutic moiety.

### DETAILED DESCRIPTION OF DRAWINGS

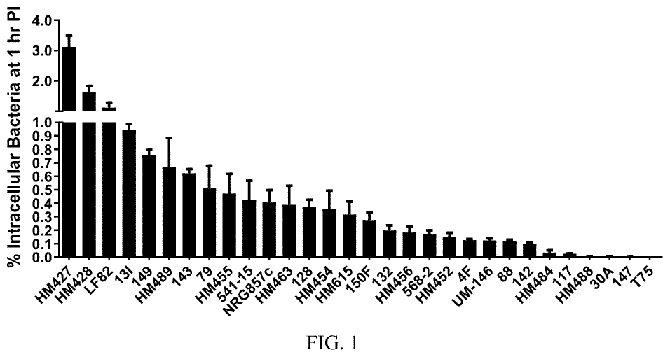

FIG. 1 is a plot of the internalization of commensal E. coli strains into Caco2 cells.

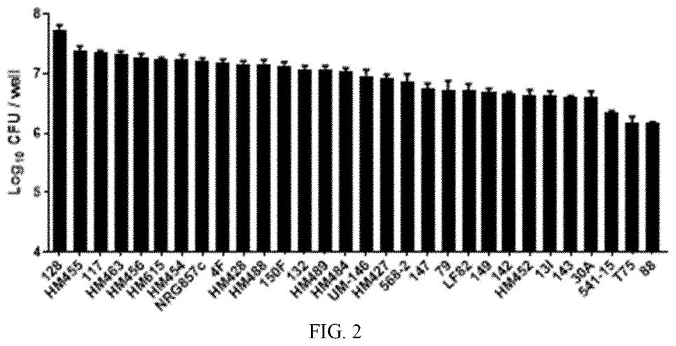

FIG. 2 is a plot of the adherence of commensal E. coli strains to Caco2 cells.

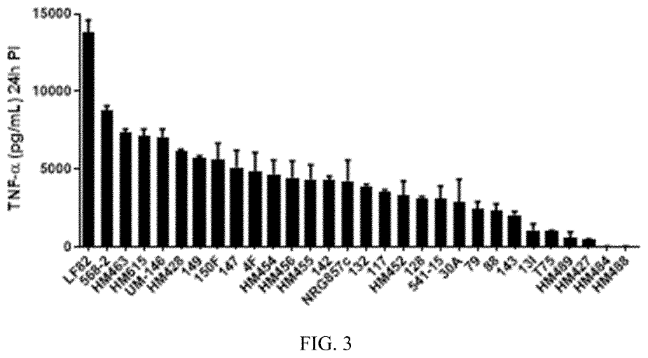

FIG. 3 is a plot of TNF-α production from J774 cells infected with commensal E. coli strains.

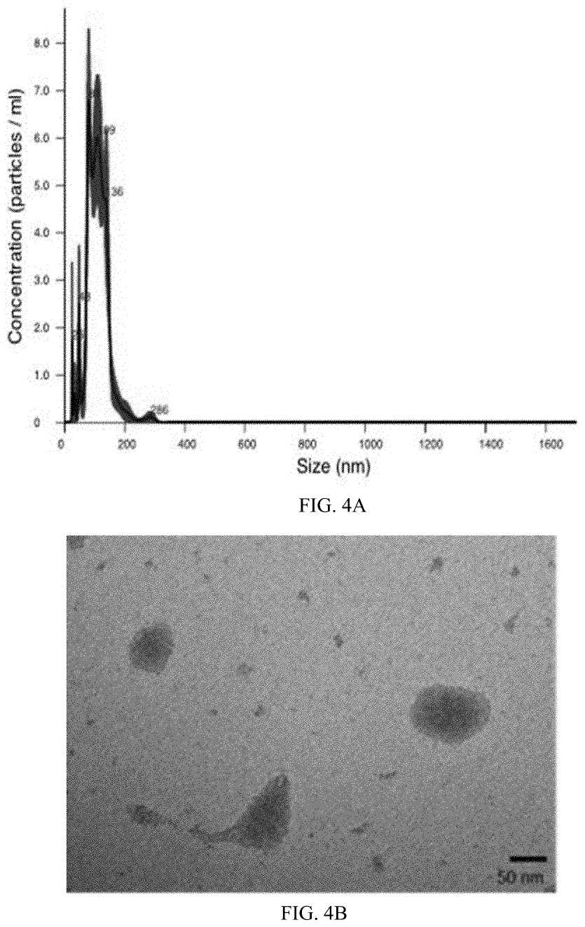

FIG. 4A is a plot of isolated OMV diameter.

FIG. 4B is a transmission electron microscopy (TEM) image of OMVs.

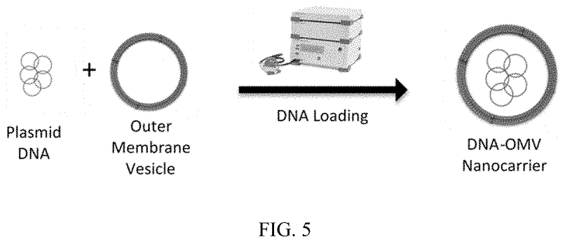

FIG. 5 is a flow chart depicting the steps to create DNA-OMV nanocarriers.

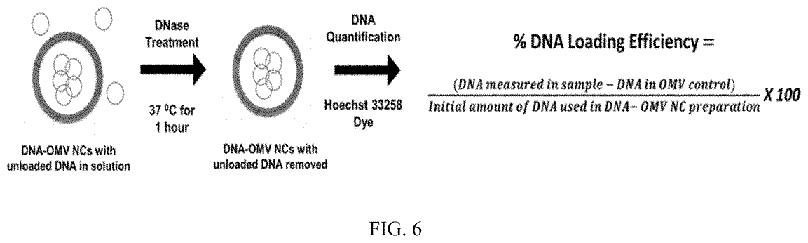

FIG. 6 is a flow chart depicting the steps to measure and calculate DNA loading efficiency.

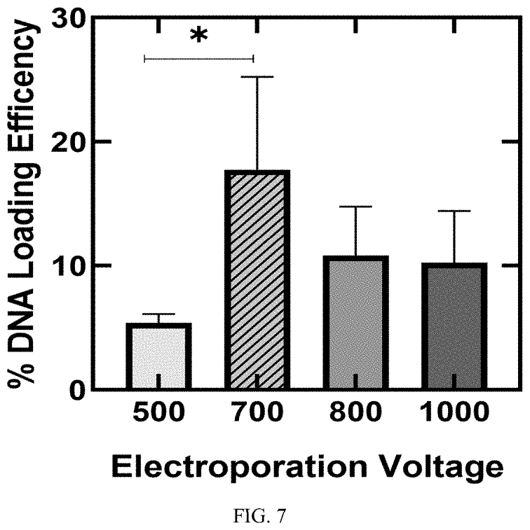

FIG. 7 is plot of the loading efficiency of DNA-OMV nanocarriers with various electroporation voltages.

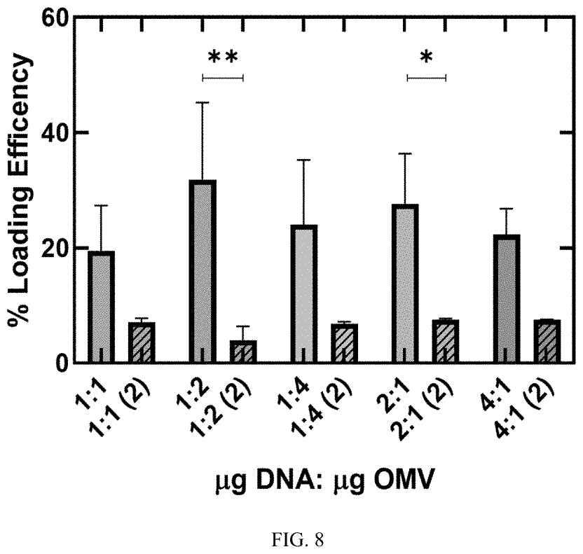

FIG. 8 is a plot of the loading efficiency of DNA-OMV nanocarriers with different DNA to OMV ratios when loaded with various numbers of electroporation pulses.

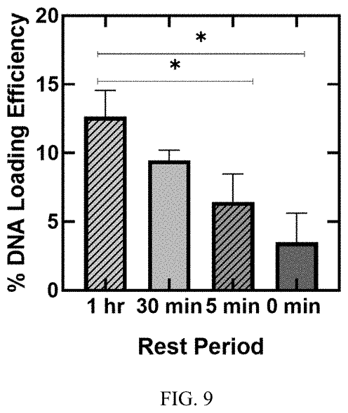

FIG. 9 is a plot of the loading efficiency of DNA-OMV nanocarriers with different rest periods after electroporation.

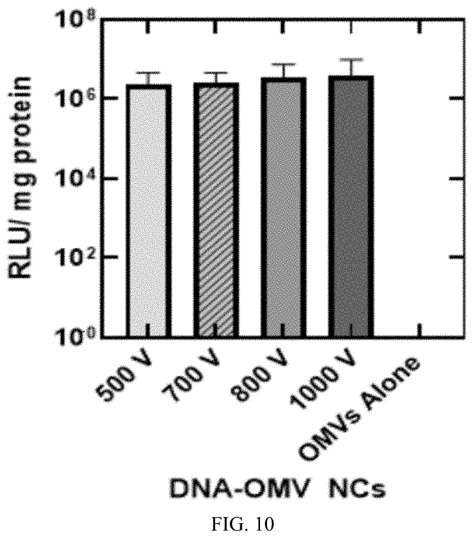

FIG. 10 is a plot of the loading efficiency of DNA-OMV nanocarriers with various electroporation voltages.

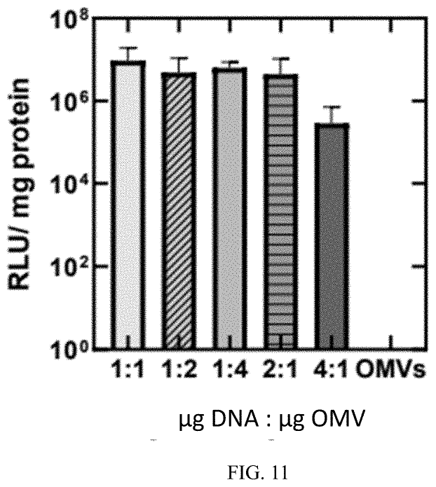

FIG. 11 is a plot of the loading efficiency of DNA-OMV nanocarriers with various DNA-to-OMV ratios.

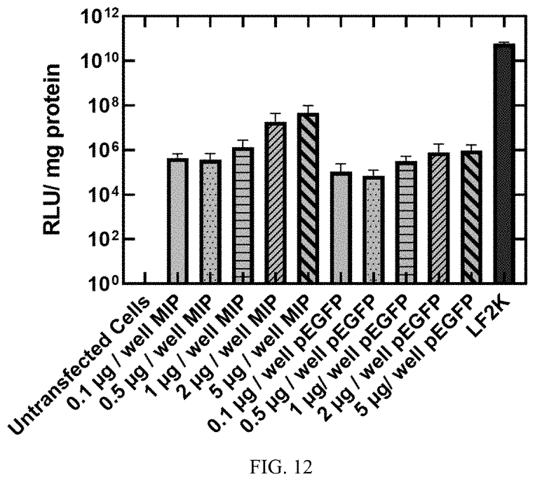

FIG. 12 is a plot of transfection of HEK 293T cells with DNA-OMV nanocarriers.

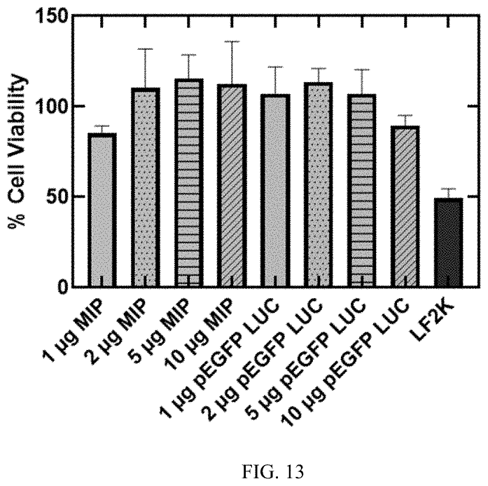

FIG. 13 is a plot of the cytotoxicity of DNA-OMV nanocarriers transfected into HEK 293T cells.

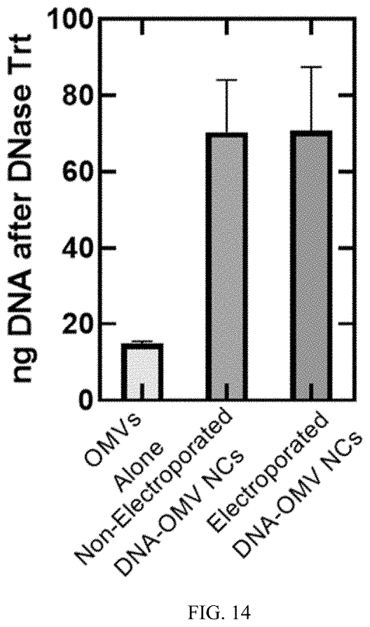

FIG. 14 is a plot of the quantification of the amount of nanograms of DNA on DNA-OMV nanocarriers after DNase treatment.

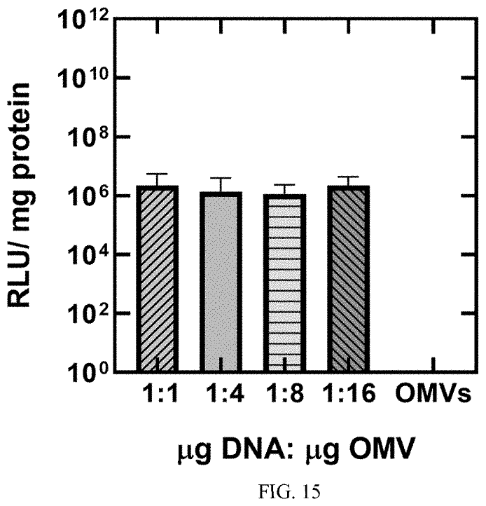

FIG. 15 is a plot of transfection success of HEK 293T cells with surface associated DNA of DNA-OMV nanocarriers at various DNA-to-OMV ratios.

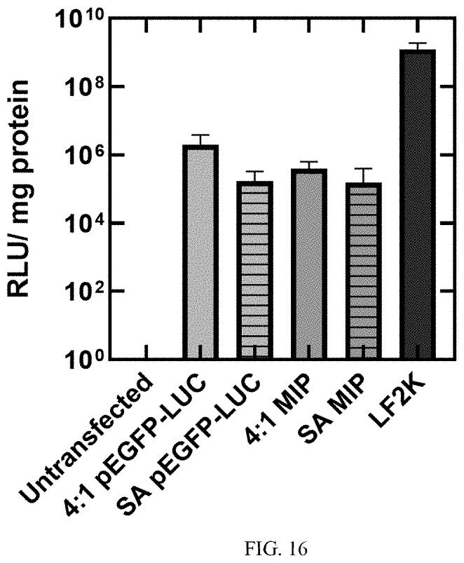

FIG. 16 is a plot of transfections success of Caco2 cells with DNA-OMV nanocarriers loaded with different DNA. pEGFP-LUC and MIP are plasmids. The MIP plasmid contains green fluorescent protein (GFP) and luciferase (LUC) reporter transgenes. LF2K is Lipofectamine 2000 and is a transfection reagent used as a control. SA=surface associated.

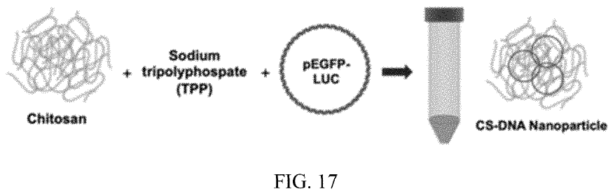

FIG. 17 is a depiction of the production of chitosan (CS)-DNA nanoparticles.

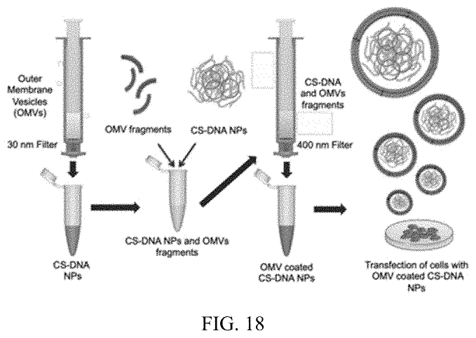

FIG. 18 is a depiction of the dual extrusion process used to coat chitosan (CS)-DNA nanoparticles (NPs) with OMVs.

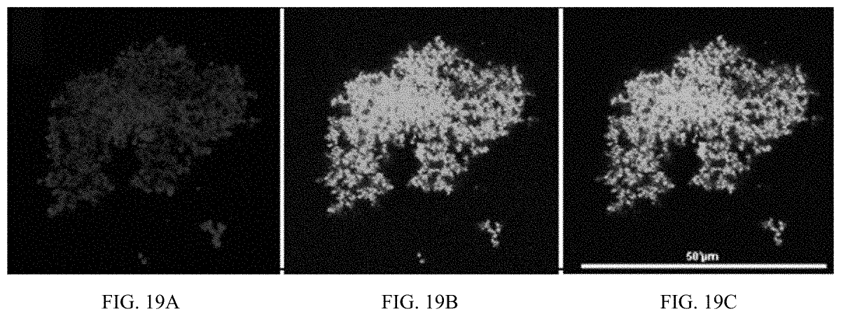

FIG. 19A is a microscopic image of forte blue labeled OMVs.

FIG. 19B is a microscopic image of FITC labeled CS-DNA NPs.

FIG. 19C is a merged microscopic image of a forte blue labeled OMBs and FITC labeled CS-DNA NPs showing co-localization of labeled OMVs and CS-DNA NPs.

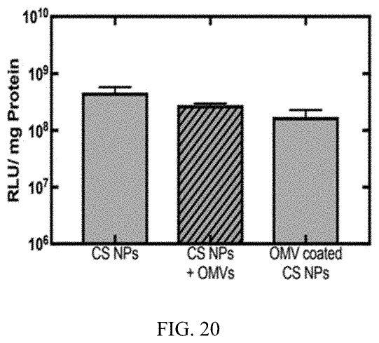

FIG. 20 is a plot of the transfection efficiency of CS-DNA nanoparticles (NPs), CS-DNA NPs with OMVs in the media and OMV coated CS-DNA NPs

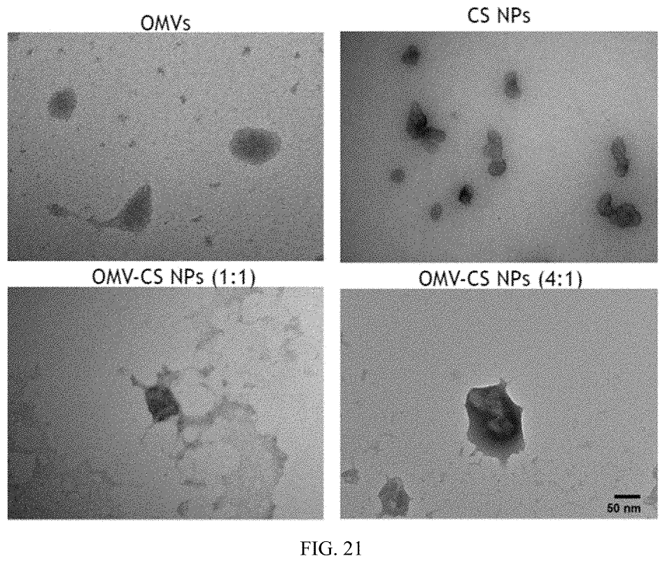

FIG. 21 is a transmission electron microscopy image of CS-OMV NPs.

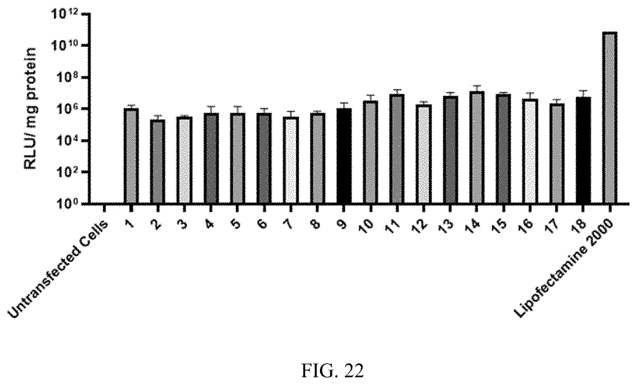

FIG. 22 is a plot of transfection efficiency of HEK 293T cells electroporated with CS-DNA NPs.

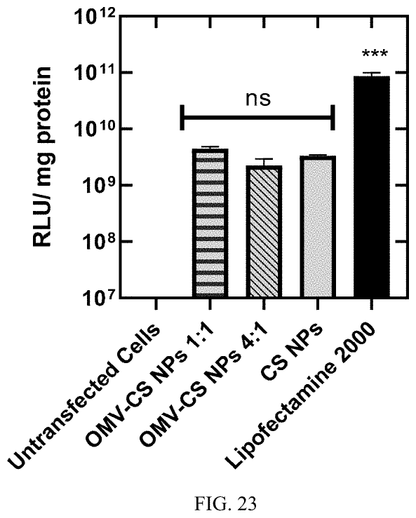

FIG. 23 is a plot of transfection efficiency of HEK293T cells with OMVs-CS NPs.

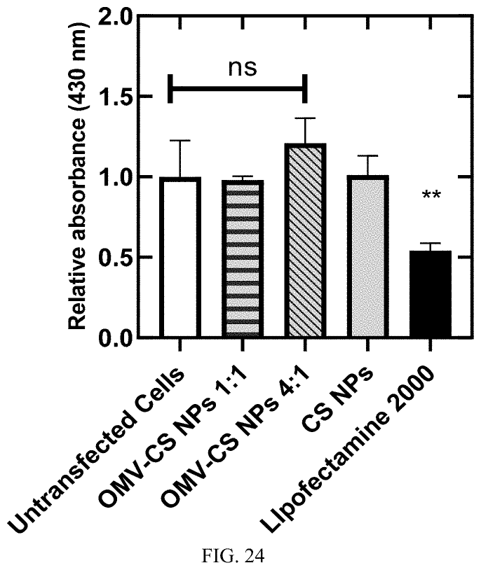

FIG. 24 is a plot of cytotoxity of OMVs-CS NPs on HEK293T cells.

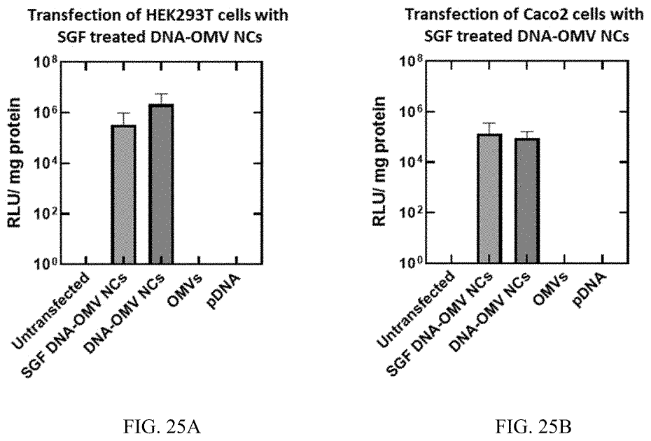

FIG. 25A is a plot of relative luminescence units (RLU) per milligram of protein of HEK293T cells transfected with simulated gastric fluid (SGF) treated DNA-OMVs (DNA-OMV NCs).

FIG. 25B is a plot of relative luminescence units (RLU) per milligram of protein of Caco2 cells transfected with SGF treated DNA-OMVs (DNA-OMV NCs).

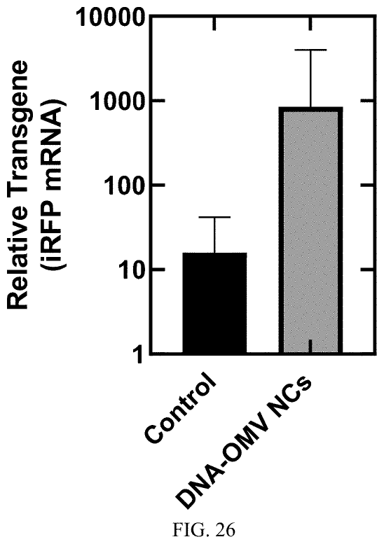

FIG. 26 is a plot of relative transgene expression from reporter plasmid (iFRP713) in iRFP mRNA assayed from mouse intestinal tissues.

### DETAILED DESCRIPTION

This document provides methods and materials involved in making and delivering a therapeutic composition. Therapeutic compositions as described herein can include, for example, a commensal bacterial outer membrane vesicle (OMV) and a therapeutic moiety.

Outer Membrane Vesicles (OMVs)

Outer membrane vesicles (OMVs) are secreted from bacteria, for example, gram-negative bacteria, via blebbing the outer membrane. While OMVs are normally associated with gram-negative bacteria, as used herein “outer membrane vesicle” or “OMV” can also refer to OMV-like vesicles produced by gram-positive bacteria. In this application, the bacteria are commensal bacteria of a subject or patient. As used herein, “commensal bacteria” refers to one or more bacterial species that are typically residents of a subject's or patient's gut. Commensal bacteria can be residents or residential bacteria of a gastrointestinal (GI) tract. As used herein, “residential bacteria” or “a residential bacterium” is defined as a microorganism that establishes itself on or in (colonizes) nonsterile parts of the body, such as the skin, nose, mouth, throat and gastrointestinal tract. Commensal bacteria typically are not pathogenic (e.g., disease causing) in healthy patients. However, OMVs isolated from commensal bacteria can be tested for cytotoxicity.

Commensal gut bacteria can have inherent immunomodulatory properties mediated by microbe-associated molecular patterns (MAMPs), including, but not limited to, lipopolysaccharide (LPS) and unmethylated CpG DNA. These same properties can be present in OMVs derived from these bacteria and can be used to customize immunomodulatory properties (e.g., tuning the OMV to the host's immune response). For example, OMVs derived from various gut commensal bacteria can provide application-specific, or tunable, immunomodulatory properties (e.g., tolerizing agent for gene therapy or adjuvant for vaccine delivery) that, in some cases, can act synergistically with a therapeutic moiety.

Commensal bacteria from which the OMVs are derived can be an intergeneric bacterial species. Commensal bacteria from which the OMVs are derived can be not intergeneric bacterial species. As used herein, “intergeneric bacteria” are bacteria formed by the deliberate combination of genetic material originally isolated from organisms of different taxonomic genera. An “intergeneric mutant” can be used interchangeably with “intergeneric bacteria.” An exemplary “intergeneric bacteria” includes bacteria containing a mobile genetic element that was first identified in bacteria in a genus different from the recipient bacterial species. Further explanation can be found, inter alia, in 40 C.F.R. § 725.3. In some cases, bacterial species can be modified to produce a higher number of OMVs of OMVs having a particular characteristic than non-modified bacterial species, for example, through genetic mutations in the parental bacteria (See, for example, Schwechheimer et al. 2014, BMC Microbiology, 14:324) or through environmental stressors (See, for example, MacDonald et al. 2013, Journal of Bacteriology, 195:13).

Commensal bacteria can be, without limitation, gram-negative bacteria. Commensal bacteria can be of a phylum selected from Bacteroidetes, Proteobacteria, Fusobacteria, or Verrucomicrobia. Commensal bacteria can be from a genus selected from Akkermansia, Bacteroides, Enterococcus, Escherichia, Klebsiella, Parabacteroides, Prevotella, Pseudomonas, Staphylococcus, Streptococcus, or Veillonella. Commensal bacteria can be Escherichia coli. E. coli strains can be selected from DH5-alpha, 541-15, 568-2, T75, LF82, 79, 88, 117, 128, 132, 142, 143, 147, 149, UM-146, HM427, HM428, HM452, HM454, HM455, HM456, HM463, HM484, HM488, HM489, HM615, 4F, 13I, 30A, 150F, NRG857c. In addition, it would be appreciated that commensal bacteria also can be gram-positive bacteria, for example, of a genera selected from Bacillus, Bifidobacterium, Lactobacillus, Leuconostoc, Pediococcus, or Streptomyces.

OMVs can survive transit through a GI tract and can be internalized into host cells. In some cases, OMVs can be internalized into intestinal epithelial cells. Internalization of OMVs by host cells, such as intestinal epithelial cells, can be tested in vitro using an internalization assay with, for example, intestinal epithelial cells, such as Caco2 intestinal epithelial cells. See, for instance, Example 1.

OMVs can have a diameter that ranges between about 10 nm to about 500 nm. For example, in some embodiments the OMVs can have a diameter that ranges between about 100 nm to about 500 nm, about 200 nm to about 500 nm, about 300 nm to about 500 nm, about 400 nm to about 500 nm, about 10 nm to about 400 nm, about 10 nm to about 300 nm, about 10 nm to about 200 nm, or about 10 nm to about 100 nm. In some embodiments, OMVs can have a diameter, for example, that ranges between about 50 nm to about 300 nm, about 50 nm to about 250 nm, about 50 nm to about 200 nm, about 50 nm to about 150 nm, about 50 nm to about 100 nm, about 100 nm to about 300 nm, about 150 nm to about 300 nm, about 200 nm to about 300 nm, or about 250 nm to about 300 nm. In some embodiments, OMVs can have a diameter that ranges between about 50 nm to about 300 nm.

In some cases, a commensal bacterial OMV fraction or commensal bacterial OMVs can be isolated, purified, or enriched from cultures of OMV-producing commensal bacteria. Purification, isolation or enrichment can be size-based. As used herein, “purified” refers to a solution in which at least 90% by mass is made up of OMVs. As used herein “isolated” refers to a solution in which the viable bacteria are at least 95% fewer than a corresponding non-isolated solution, such as a bacterial culture. As used herein, “enriched” refers to a solution that contains more OMVs or a larger percentage of the total mass of OMVs compared to a corresponding non-enriched solution, such as a bacterial culture.

OMVs can also be isolated, purified, or enriched based on the presence or absence of a marker, for example, a biomarker. Markers can be associated with, attached to, or embedded within the membrane of the OMV, and can be found on the inner or outer surface of the OMV. Alternatively, markers can indicate, for example, the removal of other bacterial components and are absent or decreased in the isolated, purified, or enriched OMV preparation. Non-limiting examples of biomarkers include the presence or increase of lipopolysaccharide or outer membrane proteins, for example, outer membrane proteins A, C, or F (OmpA, OmpC, or OmpF), and the absence or decrease of unmethylated CpG DNA. Isolation or purity of an OMV preparation can be determined, for example, by plating the OMV solution on bacterial agar plates and incubating for one or more days at an appropriate temperature to ensure a lack of bacterial growth.

It would be appreciated that OMVs derived from a pathogenic bacteria may not be suitable for use in a therapeutic composition as described herein, however, OMVs derived from a bacterial species known to stimulate an immune response (e.g., a pathobiont bacterial species or a pathogen) may be useful, for example, as an adjuvant.

### Therapeutic Moieties

In some cases, an OMV can carry a cargo, e.g., one or more therapeutic moieties. Therapeutic moieties can be carried inside, for example, encapsulated by, an OMV, or can be on the outside of, for example, attached to or associated with the surface of, an OMV. OMVs that are carrying one or more therapeutic moieties can be referred to as loaded OMVs.

Examples of therapeutic moieties that can be carried by an OMV include, without limitation, nucleic acids (e.g., DNA, plasmid DNA, siRNAs, microRNAs, mRNAs, and ncRNAs), polypeptides (e.g., enzymes), lipids, metabolites, chemicals, drugs, or small molecules. Nucleic acids can include DNA and RNA. Examples of DNA that can be carried by OMVs include DNA constructs, such as a promoter operably linked to a gene, or plasmid DNA. Examples of RNA molecules can include silencing RNAs (siRNAs), inhibitory RNAs (RNAis) microRNAs, non-coding RNAs (ncRNAs), and messenger RNAs (mRNAs). In some cases, RNA molecules that are carried within or on an OMV can inhibit or modulate expression of a polypeptide that contributes to a disease state or symptoms of a disease.

For example, a therapeutic nucleic acid, which can include therapeutic DNA or RNA, can be contained on one or more viral vectors. Such a viral vector can be loaded into and/or onto commensal bacterial OMVs and introduced into one or more host cells, such as GI tract epithelial cells. Any appropriate DNA virus vector in or on a commensal bacterial OMV can be used to deliver and/or express one or more nucleic acids encoding one or more therapeutic polypeptides to a mammal, such as a human. A viral vector can be derived from a positive-strand virus or a negative-strand virus. In some cases, a viral vector can be a chimeric viral vector. In some cases, a viral vector can infect dividing cells. Examples of virus-based vectors that can be used to deliver nucleic acid encoding a therapeutic polypeptide to a mammal include, without limitation, virus-based vectors based on adenoviruses (AVs), adeno-associated viruses (AAVs), Herpes simplex virus (HSV), cytomegalovirus (CMV), vesicular stomatitis virus (VSV), measles, and Epstein-Barr virus (EBV).

Additionally, therapeutic nucleic acids can be introduced into one or more host cells, such as GI tract epithelial cells, using one or more non-viral vectors. When a vector used to deliver and/or express one or more therapeutic nucleic acids encoding one or more therapeutic polypeptides to a mammal, such as a human, is a non-viral vector, any appropriate non-viral vector can be used. In some cases, a non-viral vector can be an expression plasmid.

In addition to one or more therapeutic nucleic acids, a vector (e.g., a viral vector, a non-viral vector, or a plasmid) can contain regulatory elements operably linked to the therapeutic nucleic acid. Such regulatory elements can include promoter sequences, enhancer sequences, response elements, signal peptides, internal ribosome entry sequences, polyadenylation signals, terminators, or inducible or suppressible elements that modulate expression (e.g., transcription) or activity (e.g., translation) of a therapeutic moiety such as a therapeutic nucleic acid or a therapeutic polypeptide or protein. The choice of element(s) that may be included in a vector depends on several factors, including, without limitation, inducibility, targeting, and the level of expression desired. A promoter can be constitutive or inducible (e.g., in the presence of tetracycline), and can affect the expression of one or more therapeutic nucleic acids encoding a polypeptide in a general or tissue-specific manner. Examples of promoters that can be used to drive expression of one or more therapeutic nucleic acids, without limitation, are U6, H1, and T7 promoters. As used herein, “operably linked” refers to positioning of a regulatory element in a vector relative to a nucleic acid in such a way as to permit or facilitate expression of the nucleic acid.

The nucleic acid can encode a protein that is expressed in the host cell, such as GI tract or intestinal epithelial cells. The protein can be a therapeutic protein, a transcriptional activator, or a transcriptional repressor. As used herein, the term “transcriptional activator” refers to a polypeptide which can activate or increase transcription of a specific promoter or set of promoters necessary for the initiation of transcription of the polypeptide encoded by the DNA sequence to which it is operably linked. As used herein, the term “transcriptional repressor” refers to a polypeptide which can inactivate or decrease transcription of a specific promoter or set of promoters necessary for the initiation of transcription of the polypeptide encoded by the DNA sequence to which it is operably linked. The therapeutic protein is selected from a cytokine, an antigen, an antibody, a biologic, a growth factor, a differentiation factor, an enzyme, or an immune modulating factor. Therapeutic proteins can alleviate, lessen, reduce, or abolish symptoms of disease, for example, inflammatory bowel disease, ulcerative colitis, Crohn's disease, colon cancer, irritable bowel syndrome, lactose intolerance, celiac disease.

In some cases, the therapeutic moiety can be a therapeutic protein or an antibody.

In some cases, the therapeutic moiety can be a drug, such as an antibiotic, a biologic, a steroid, a chemotherapeutic, an immunosuppressant, and/or a vaccination protein.

In some cases, the therapeutic moiety can include gene editing components (e.g., one or more components of a clustered regularly interspaced short palindromic repeats (CRISPR)/Cas system or components of a transcription activator-like effector nucleases (TALENS) system).

Depending on the therapeutic moiety used, the OMVs can be selected and/or engineered (e.g., tuned) for a desired result (e.g., pro-inflammatory or anti-inflammatory). In some cases, suitable OMVs are those that have an anti-inflammatory effect (e.g., decrease the immune response of the host, for example, when used to treat symptoms associated with inflammation, such as treating symptoms associated with inflammatory bowel disease). In some cases, suitable OMVs are those that have a pro-inflammatory effect (e.g., increase the immune response of the host, for example, when used with a vaccine).

### Methods of Making and Using Therapeutic OMVs

OMVs can be obtained using any number of methods. OMVs can be generated using any number of methods such as, without limitation, by filtering a bacterial culture that contains OMV-producing bacteria using a variety of filtration methods (e.g., tangential flow filtration, ultrafiltration, ultracentrifugation, ultracentrifugation with sucrose gradients, size exclusion chromatography (SEC)), or by using, e.g., an ExoBacteria OMV Isolation kit (System Biosciences).

Any appropriate method can be used to detect OMVs, the presence or absence of an OMV population, and/or the presence or absence of an OMV population within a sample (e.g., a sample obtained from bacterial culture). For example, cytometry methods (e.g., flow cytometry such as cell sorting), spectrometry methods, antibody dependent methods (e.g., enzyme-linked immunosorbent assays (ELISAs), immunoprecipitation, immunoelectrophoresis, filtration methods, and/or western blotting methods can be used to detect OMVs, the presence or absence of an OMV population, and/or the presence or absence of an OMV population within a sample (e.g., a sample obtained from a bacterial culture). Representative methods in which the presence of OMVs and/or the amount of OMVs were determined are described in, for example, Horstman and Kuehn 2000 (doi: 10.1074/jbc.275.17.12489), Bai et al. 2014 (doi: 10.1128/IAI.01416-13) and Carvalho et al. 2019 (doi: 10.1111/cei.13301).

Any appropriate method can be used to load one or more therapeutic moieties, for example nucleic acid molecules, in or on commensal bacterial OMVs, such as purified, isolated, or enriched commensal bacterial OMVs. For example, OMVs (e.g., isolated, purified, or enriched OMVs) can be electroporated in the presence of one or more therapeutic moieties (e.g., nucleic acid moieties such as DNA molecules or plasmid DNA). Additionally, therapeutic moieties can by adsorbed to the surface of an OMV by incubation of the OMVs with the therapeutic moieties in solution, or therapeutic moieties can be loaded into OMVs via extrusion by combining the moieties with OMVs in solution and then extruding the solution through a filter with a pore size smaller than the diameter of the OMVs. The extrusion coating process utilizes a filter that has a pore size smaller than vesicle diameter to disrupt vesicle membranes into membrane fragments, for example, via shear force. After the membrane fragments have passed through the filter, they can spontaneously reform into vesicles due to the hydrophobic effect.

Electroporation can use voltages between about 250V to about 2000V. For example, between about 300V to about 2000V, between about 400V to about 2000V, between about 500V to about 2000V, between about 600V to about 2000V, between about 700V to about 2000V, between about 800V to about 2000V, between about 900V to about 2000V, between about 1000V to about 2000V, between about 200V to about 1900V, between about 200V to about 1900V, between about 200V to about 1800V, between about 200V to about 1700V, between about 200V to about 1600V, between about 200V to about 1500V, between about 200V to about 1400V, between about 200V to about 1300V, between about 200V to about 1200V, between about 200V to about 1100V, between about 200V to about 1000V, between about 200V to about 900V, between about 200V to about 800V, between about 200V to about 700V, between about 200V to about 600V, between about 200V to about 500V, between about 400V to about 1100V, between about 500V to about 1100V, between about 600V to about 1100V, between about 700V to about 1100V, between about 800V to about 1100V, between about 900V to about 1100V, between about 500V to about 1000V, between about 500V to about 900V, between about 500V to about 800V, between about 500V to about 700V, or between about 600V to about 800V. Electroporation can use one pulse or multiple pulses. For example, electroporation can use one pulse, two pulses, or three pulses.

Other methods also can be used to load OMVs with a therapeutic moiety as described herein. For example, sonication, saponin treatment, incubation with OMVs, or extrusion can be used to load a therapeutic moiety into or onto OMVs. Without wishing to be bound by theory, any method that reversibly permeabilizes a bacterial membrane or induces uptake of DNA, RNA, proteins, chemicals, or small molecules can be used.

Electroporation or other methods of introducing the therapeutic moiety into or onto an OMV can use various ratios of therapeutic moiety (e.g., DNA) to commensal bacteria OMVs as measured by weight, such as micrograms. In some cases, about 1 microgram therapeutic moiety to about 1 microgram OMVs, about 1 microgram therapeutic moiety to about 2 micrograms OMVs, about 1 microgram therapeutic moiety to about 3 micrograms OMVs, about 1 microgram therapeutic moiety to about 4 micrograms OMVs, about 2 microgram therapeutic moiety to about 1 micrograms OMVs, about 3 microgram therapeutic moiety to about 1 micrograms OMVs, about 4 microgram therapeutic moiety to about 1 micrograms OMVs. For example, about 1 microgram DNA to about 1 microgram OMVs, about 1 microgram DNA to about 2 micrograms OMVs, about 1 microgram DNA to about 3 micrograms OMVs, about 1 microgram DNA to about 4 micrograms OMVs, about 2 microgram DNA to about 1 micrograms OMVs, about 3 microgram DNA to about 1 micrograms OMVs, about 4 microgram DNA to about 1 micrograms OMVs. In some cases, electroporation can use 1 microgram of therapeutic moiety, such as DNA, to 2 micrograms isolated, purified, or enriched commensal bacteria OMVs. In some instances, a therapeutic moiety can be combined with OMVs at a ratio that would result in about 1, or less than about 1, therapeutic moiety per OMV.

In some cases, commensal bacteria OMVs loaded with one or more therapeutic moieties can be formulated together with one or more pharmaceutically acceptable carriers (e.g., additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

In some cases, any of the therapeutic compositions described herein, such as commensal bacterial OMVs loaded with therapeutic moieties, can be delivered to an individual, for example, a mammal (e.g., a human), or any other type of animal, via oral delivery, intraperitoneal administration, intranasal administration, intravenous administration, intrathecal administration, intracerebral administration, intraparenchymal administration, or direct injection (e.g., into a particular organ or tissue). In some cases, any of the therapeutic compositions described herein, such as commensal bacterial OMVs loaded with therapeutic moieties can be delivered to an individual via oral delivery, intranasal administration, or rectal administration (e.g., an enema). In some cases, the therapeutic composition can be formulated as a tablet, a suspension, a capsule, or other suitable formulation for oral delivery. Also, the therapeutic composition can be lyophilized or, for example, be formulated as a tablet or capsule. In some cases, the therapeutic composition can be formulated as an enema. Non-limiting examples of individuals that can benefit from the methods and compositions described herein include, for example, mammals (e.g., humans and non-human primates such as monkeys as well as dogs, cats, horses, cows, pigs, sheep, llamas, mice, rats, guinea pigs, and rabbits). In addition to humans, “subjects” or “individuals” referred to herein include, without limitation, companion animals, zoo animals, livestock or farm animals, and exotic or rare animals.

Any of the therapeutic compositions described herein can be administered to an individual or subject. Any of the therapeutic compositions described herein can be administered once or can be administered multiple times a day, multiple times a week, multiple times a month, or multiple times a year. In some cases, the therapeutic compositions described herein can be administered daily, weekly, monthly, or yearly. In some cases, any of the therapeutic compositions described herein can be administered to an individual a plurality of times. In some cases, any of the therapeutic compositions can exhibit a delayed time release or can be formulated for a timed-release of the one or more therapeutic moieties. In some cases, any of the therapeutic compositions can have an extended release over an hour, multiple hours, multiple days, or multiple weeks.

Any of the methods of delivering any of the described therapeutic moieties or therapeutic compositions can further comprise monitoring the therapeutic moiety or monitoring the individual or subject's response to the therapeutic moiety.

### EXAMPLES

### Section A: OMVs

### Example 1. Screening Commensal Bacteria for OMVs with High Adherence and Low Cytokine Production

A library of 30 commensal E. coli strains isolated from humans was screened for adherence and cytokine production using in vitro techniques. Different strains display different interactions with host cells. Commensal E. coli strains had differential internalization into Caco2 cells (FIG. 1), adhered to intestinal epithelial cells and elicited differential cytokine production in cell lines in a strain-specific manner (FIGS. 2-3). Outer membrane vesicles possessed similar properties to the bacterial from which they were isolated since they have a similar composition as the bacterial outer membrane. E. coli strain HM488 was selected for further testing because it is adherent and does not elicit high levels of pro-inflammatory cytokine production from J447 cells in vitro.

OMVs were isolated from Escherichia coli DH5-alpha culture using ExoBacteria OMV Isolation Kit (SystemsBiosciences). Size confirmation of OMVs was performed using a NanoSight NS3000 (Malvern) and demonstrated that OMVs had a mean diameter of 110.2±4.2 nm. A bicinchoninic acid (BCA) assay was used to determine the yield of OMVs (e.g., μg OMV protein/mL) (ThermoFisher) (FIG. 4A), and TEM was used to image isolated OMVs using a Hitachi 1-17500 TEM (FIG. 4B).

### Section B: DNA

### Example 2. Loading OMVs with Plasmid DNA Using Electroporation

OMVs can be loaded with exogenous plasmid DNA using electroporation (FIG. 5). Plasmid DNA (pDNA; pEGFP-LUC) encoding for an enhanced green fluorescent and a luciferase fusion protein was loaded into isolated OMV via electroporation using a BioRadGene Pulser XCell.

Parameters for high loading efficiency were determined. Loading efficiency of the DNA-OMV nanocarriers (also called DNA-OMVs) was determined by applying DNase treatment after electroporation for 30 min at 37° C. DNA within the DNA-OMV nanocarriers was quantified using Hoechst 33258 dye. Loading efficiency of the DNA-OMV nanocarriers was calculated by dividing the amount of encapsulated DNA in DNA-OMV nanocarriers after DNase treatment by the initial amount of DNA in the DNA-OMV nanocarrier preparation. These experiments are schematically shown in FIG. 6.

While holding pulse length (exponential decay) and resistance (50 μF) constant, electroporation voltages were varied between 500-1000 V to optimize DNA loading efficiency. These results are shown in FIG. 7. Next, the μg DNA: μg OMV protein ratio was varied between 1:1 and 1:4 while holding electroporation parameters constant to determine the impact of the ratio on DNA loading efficiency. These results are shown in FIG. 8. Loading efficiency of DNA-OMV nanocarriers was also affected by the length of the rest period after electroporation. As shown in FIG. 9, a 1-hour rest period produced the highest loading efficiency.

### Example 3: Surface Associated DNA Remains after DNase Treatment

To determine the location of loaded DNA in our DNA-OMV NCs, a PicoGreen assay was employed to measure externally or surface associated DNA since PicoGreen cannot cross the cell membrane unlike Hoechst 33258 dye.

To determine the amount surface loaded DNA in electroporated DNA-OMV NCs a ratio of 1:1 DNA: OMV was used, and electroporation was performed at 700 V. DNase treatment was then used to determine how much DNA remained surface associated and was not floating in solution (FIG. 14). To examine if we could load the surface of OMVs with plasmid DNA via simple incubation of OMVs and DNA together, a 1:1 DNA: OMV mixture that was not electroporated was used (FIG. 14). Both the surface associated and electroporated DNA-OMV NCs had higher amounts of surface associated DNA when compared to native OMVs alone. These results indicate that surface associated DNA is present on electroporated OMVs, that OMVs can be loaded with surface associated DNA via incubation at room temp with plasmid DNA, and that surface associated DNA can survive DNase treatment (FIG. 15). In addition, transfection efficacy of surface associated DNA on OMVs was evaluated with HEK293T cells. A volume of DNA-OMV nanocarriers (also called DNA-OMVs) containing 0.5 μg of surface associated DNA were applied to cells and transgene production was evaluated 24 hours later. FIG. 16 shows that surface associated DNA on OMVs is capable of transfecting HEK 293T cells. Transfection efficiency of DNA-OMV NCs (loaded via electroporated or surface association) was also evaluated in Caco2 intestinal epithelial cells (FIG. 16). A volume of DNA-OMV nanocarriers containing 0.5 μg of DNA were applied to cells and transgene production was evaluated 24 hours later. Electroporated and surface-associated DNA-OMV NCs showed similar transfection efficiencies in Caco2 cells (FIG. 16).

### Section C: Making Therapeutic OMVs

### Example 4. Transfecting HEK293T Cells with DNA-Loaded OMVs

Transfection efficacy of DNA-OMV nanocarriers (also called DNA-OMVs) was investigated by transfecting HEK 293T cells with DNA-OMV nanocarriers with equal amounts of DNA delivered to cells across all treatments. Non-loaded OMVs were used as a negative control for transfection. Transfection results were quantified by measuring transgenic luciferase activity and normalizing to total cellular protein. A volume of DNA-OMV nanocarriers containing 0.5 pg of pEGFP-LUC plasmid were incubated with cells for 24 hours. Cells were then lysed and cell lysates were measured for transgene expression using the luciferase assay system (Promega). Luciferase activity was measured in relative light units (RLUs), which were normalized to total protein content measured by the BCA protein assay (Pierce). DNA-OMV nanocarriers were able to mediate transfection within HEK 293T cells (FIG. 10); however, the different electroporation voltages tested did not significantly impact DNA-OMV nanocarrier transfection.

In another experiment, DNA-OMV nanocarriers formulated with two electroporation pulses and used to test the transfection efficacy of HEK293T cells with varying rations of DNA:OMVs. As shown in FIG. 11, the DNA:OMV ratio did not impact transfection efficiency.

In another experiment, the effect of the amount of DNA and MIP/pEGFP on the transfection of HEK 293T cells was tested (FIG. 12). Cytotoxicity of DNA-OMVs on cell viability was also tested using a WST-8 assay and demonstrated low to no cytotoxicity of DNA-OMV NCs on HEK 293T cells (FIG. 13).

### Example 5. Formation of Chitosan (CS)-DNA Nanoparticles (NPs)

In this example, a dual biomaterial particle delivery system composed of chitosan (CS) and outer membrane vesicles (OMVs) is described. Methods to coat CS-DNA nanoparticles (CS-DNA NPs) with OMVs to survive gastric transit are described.

CS-DNA NPs were formed with ultra-pure chitosan, sodium tri polyphosphate (TPP) and plasmid DNA (pEGFP-LUC), which encodes for an enhanced green fluorescent and luciferase fusion protein (FIG. 17). CS-DNA NPs were characterized for size and surface charge. Commensal Escherichia coli HM488, isolated from a healthy human, was grown. OMVs were then isolated using an ExoBacteria OMV isolation kit and sized using a Malvern NanoSight NS 3000. Strain HM488 was chosen from a library of over 30 commensal E. coli strains due to its ability to adhere to Caco2 epithelial cells in vitro and its failure to induce disease in a mouse model of colitis. CS-DNA NPs were coated with OMVs via a two-step extrusion process (FIG. 18). OMVs were first extruded through a 30 nm filter to disrupt membrane integrity using shear force. OMV fragments and CS-DNA NPs were then combined and extruded through a 400 nm filter to facilitate reformation of the OMV fragments around the CS-DNA NPs.

OMV-coated CS-DNA NPs obtained at the end of the extrusion process were analyzed with confocal microscopy. Co-localization of forte blue labeled OMVs and FITC labeled CS-DNA NPs verified successful coating. OMVs successfully isolated from commensal E. coli were 110.2±4.2 nm. CS-DNA NPs were 166.4±1.3 nm. No significant changes in nanoparticle size or transfection efficiency were observed before and after extrusion. Co-localization of labeled OMVs and CS-DNA NPs verified assembly of OMV-coated CS-DNA NPs after the dual extrusion process (FIG. 19A-19C). Images were obtained using a Cytation at 40× objective.

After characterization, OMV-coated CS-DNA NPs were used to transfect the human embryonic kidney cell line HEK 293T. Transfection results were quantified by measuring transgenic luciferase activity and normalizing to total cellular protein. Transfection levels with CS-DNA NPs, CS-DNA NPs with OMVs in the medium and OMV-coated CS-DNA NPs (FIG. 20) were all similar, indicating OMVs do not inhibit transfection, even given their bacterial origin.

### Example 6. Formation of Chitosan (CS)-DNA Nanoparticles (NPs)

High molecular weight chitosan-DNA nanoparticles (CS-DNA NPs) were formed via ionic gelation with 0.5 mg/mL high molecular weight chitosan, CS:TPP 1:4, CS: DNA 10. Size confirmation of CS NPs was performed using Dynamic Light Scattering (Malvern) and TEM (FIG. 21). All images were taken at 50,000× magnification. A ninhydrin assay was used to quantify CS NP μg/mL (Sigma). Molecular weight of chitosan was 310-375 kDa and >75% of the chitosan was deacetylated.

The extrusion method used to coat chitosan-coated DNA nanoparticles with OMVs was optimized by using different ratios of CS-DNA NPs:OMVs. Ratios were 1:1, 1:2, and 1:4 CS-DNA NPs: OMVs.

### Example 7. OMV Coating does not Impede CS NP-Mediated Transfection and is not Cytotoxic

HEK 293T cells were seeded in 96-well plates and transfected with OMV-CS NPs or CS NPs 18 hours after seeding. At 24 hours after seeding, cells were assayed to quantify luciferase activity and to quantify protein in milligrams to determine transfection efficiency (FIG. 22). No significant difference was observed between OMV coated OMV-CS NPs and “naked” CS NPs, indicating that the OMV coating does not impede CS NP mediated transfection in HEK 293T cells (FIG. 23). Cytotoxicity of OMV-CS NPs on cell viability was also tested using a WST-8 assay and demonstrated low to no cytotoxicity on HEK 293T cells (FIG. 24).

### Section D: Using Therapeutic OMVs

### Example 8. Transgene Expression In Vitro Mediated by Delivery of DNA-OMVs to HEK293T and Caco2 Cells

In this example, in vitro transgene expression was determined in HEK293T cells and Caco2 cells transfected with OMVs loaded with plasmid containing a fusion protein of an enhanced gene fluorescence and a luciferase gene (pEGFP-LUC).

Formation of DNA-OMVs: DNA-OMVs were formulated using a DNA:OMV ratio of 1:1. OMVs were loaded via electroporation (700 V, 50 μF, exponential function) using a BioRad Gene Pulser Xcell system. Plasmid DNA encoding for an enhanced green fluorescent and a luciferase fusion protein (pEGFP-LUC) was loaded into OMVs and used to assess transgene expression in vitro.

Simulated Gastric Fluid Incubation: DNA-OMVs, formed by the loading method described above, were exposed to simulated gastric fluid (SGF; 0.034 M NaCl, 0.085 M HCl, 3.2 g/L pepsin, pH 3) before being used to transfect HEK293T and Caco2 cells. A volume of DNA-OMVs containing 3 μg of loaded pEGFP-LUC was incubated in simulated gastric fluid (SGF) fluid for 60 min at 37° C. and then collected by tangential flow filtration. SGF-treated DNA-OMVs were then resuspended in PBS and delivered to Caco2 and HEK 293T cells.

Transfection of cells in vitro: DNA-OMVs were used to transfect the human embryonic kidney cell line HEK 293T and the Caco2 human intestinal epithelial cell line with equal amounts of DNA delivered to cells across all treatments. Non-loaded OMVs and naked plasmid DNA (pDNA) were used as a negative control for transfection. Transfection results were quantified by measuring transgenic luciferase activity and normalizing to total cellular protein.

Cells were transfected with DNA-OMV NCs after simulated gastric fluid incubation. DNA-OMV NCs (also called DNA-OMVs) loaded with plasmid DNA encoding for an enhanced green fluorescent and a luciferase fusion protein were incubated in SGF for 60 minutes before being used to transfect HEK293T cells (FIG. 25A) or Caco2 cells (FIG. 25B). DNA-OMV NCs that were not subjected to SGF incubation were used to determine if SGF incubation reduced DNA-OMV NC mediated transgene expression. Untransfected cells, cells treated with unloaded OMVs and cells treated with naked pDNA were used as controls. DNA-OMV NCs incubated in SGF mediated transfection in HEK 293T and Caco2 cells at levels similar to those mediated by DNA-OMV NCs not exposed to SGF. These results indicate that DNA-OMV NCs maintain transfection competence after incubation in SGF, which mimics in vivo gastric conditions (temperature, pH and enzymes).

### Example 9. Transgene Expression In Vivo Mediated by Delivery of DNA-OMV to Mice

In this example, in vivo transgene expression was determined from mouse intestinal tissue samples after mice were orally gavaged with DNA-OMVs (also called DNA-OMV NCs) carrying the reporter plasmid, iFRP713, described in Example 7.

Formation of DNA-OMV NCs: DNA-OMV NCs were formulated using a DNA:OMV ratio of 1:1. OMVs were loaded via electroporation (700 V, 50 exponential function) using a BioRad Gene Pulser Xcell system. The reporter plasmid containing the sequence for a near-infrared fluorescent protein (iRFP713, Addgene #45468) was loaded into the OMVs and used to assess transgene expression in vivo.

Experimental Samples: 1 control mouse, 4 intestinal samples collected, 3 DNA-OMV NCs treated mice, 4 samples collected per mouse. Intestinal tissues were collected from both the small intestine and colon. Samples were snap frozen and stored at −80° C.

RT-PCR: RNA was extracted from the tissues using an miRNeasy tissue/cells kit (Qiagen) following the manufacturer's protocol and cDNA was obtained from the extracted RNA using a RT2 first strand kit (Qiagen) following the manufacturer's protocol. Power SYBR green master mix (Applied Biosystems) was used in the PCR reaction according to the manufacturer's instructions. Relative fold gene expression of the samples was calculated using the 2^{-ΔΔCt} method using 18S and glyceraldehyde-3-phosphate dehydrogenase (GADPH) as housekeeping genes. Fluorescence was measured using RT-PCT, with a forward primer with the sequence of 5′GCAAGAGGTGCGGAAGATTA (SEQ ID NO: 1), and a reverse primer with the sequence of 5′GGATGGCGAAGCTAAGATCAA (SEQ ID NO: 2).

Transgene expression mediated by delivery of plasmid DNA (pDNA) via DNA-OMV NCs was assayed in vivo. DNA-OMV NCs loaded with a reporter plasmid (iFRP713) were orally gavaged into mice at a dose of 100 μg/mouse. After 24 hours, mice were euthanized and intestinal tissues were harvested. Expression of the iRFP transgene in intestinal tissue samples was quantified using RT-PCR and normalized to control mice that received no DNA-OMV NCs. Expression of the iRFP transgene in intestinal tissue samples was quantified using RT-PCR and normalized to housekeeping genes. Oral gavage with DNA-OMV NCs in mice resulted in expression of the iRFP transgene in intestinal tissues. Detection of the iRFP transgene in intestinal tissues indicates successful delivery and expression of the pDNA payload in DNA-OMV NCs in vivo. See FIG. 26.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A therapeutic composition comprising:
a commensal bacterial outer membrane vesicle (OMV); and
a therapeutic moiety.

2. The therapeutic composition of claim 1, wherein the commensal bacterial OMV is from a bacterium that is a resident of the gut, a resident bacterium of the gut that is not typically pathogenic, a bacterium that is not intergeneric, or a gram-negative bacterial species.

3. The therapeutic composition of claim 1, wherein the commensal bacterial OMV is from a bacterial genera selected from Akkermansia, Bacteroides, Enterococcus, Escherichia, Klebsiella, Parabacteroides, Prevotella, Pseudomonas, Staphylococcus, Streptococcus, or Veillonella.

4. The therapeutic composition of claim 3, wherein the commensal bacterial OMV is an Escherichia coli bacterial species selected from DH5-alpha, 541-15, 568-2, T75, LF82, 79, 88, 117, 128, 132, 142, 143, 147, 149, UM-146, HM427, HM428, HM452, HM454, HM455, HM456, HM463, HM484, HM488, HM489, HM615, 4F, 13I, 30A, 150F, NRG857c.

5. The therapeutic composition of claim 1, wherein the commensal bacterial OMV has a diameter of about 50 nm to about 300 nm.

6. The therapeutic composition of claim 1, wherein the commensal bacterial OMV is a purified or isolated commensal bacterial OMV.

7. The therapeutic composition of claim 1, wherein the commensal bacterial OMV is enriched compared to the commensal bacterial culture.

8. The therapeutic composition of claim 1, wherein the therapeutic moiety is selected from a nucleic acid, a protein, a chemical, a drug, or a small molecule.

9. The therapeutic composition of claim 8, wherein the nucleic acid encodes a protein selected from a therapeutic protein, a transcriptional activator, or a transcriptional repressor.

10. The therapeutic composition of claim 9, wherein the therapeutic protein is selected from a cytokine, an antigen, an antibody, a biologic, a growth factor, an enzyme, a differentiation factor, an immune modulating factor, or a vaccination protein.

11. The therapeutic composition of claim 8, wherein the drug is an antibiotic, a biologic, a steroid, a chemotherapeutic, or an immunosuppressant.

12. The therapeutic composition of claim 1, further comprising a pharmaceutically acceptable carrier.

13. The therapeutic composition of claim 1, wherein the therapeutic composition is formulated as a tablet, suspension, a capsule, or other suitable formulation for oral delivery.

14. A method of making a therapeutic composition, comprising the steps of:
a) providing a commensal bacterial outer membrane vesicle (OMV); and
b) introducing a therapeutic moiety into and/or onto the commensal bacterial OMV.

15. The method of making a therapeutic composition of claim 14, wherein the providing step comprises filtering or centrifuging a culture of a commensal bacterial species to obtain the OMVs.

16. The method of making a therapeutic composition of claim 14, wherein the introducing step comprises electroporation, surface adsorption, or extrusion.

17. The method of making a therapeutic composition of claim 14, further comprising the step of measuring the average diameter of the OMVs.

18. A method of delivering a therapeutic moiety to an individual, the method comprising:
administering the therapeutic composition of claim 1 to an individual in need thereof.

19. The method of delivering a therapeutic moiety to an individual of claim 18, wherein the individual suffers from inflammatory bowel disease, ulcerative colitis, Crohn's disease, colon cancer, irritable bowel syndrome, lactose intolerance, or celiac disease.

20. The method of delivering a therapeutic moiety to an individual of claim 18, wherein the individual receives a nucleic acid-based vaccination.

21. The method of delivering a therapeutic moiety to an individual of claim 18, further comprising monitoring the therapeutic moiety.
